(19) United States
(12) Patent Application Publication
(10) Pub. No.: US 2021/0260074 A1
(43) Pub. Date: Aug. 26, 2021

(54) SEIZURE CONTROL COMPOSITIONS AND METHODS OF USING SAME

(71) Applicant: Marsh and Wang Medical Systems, LLC, Peoria, AZ (US)

(72) Inventor: Steven T. Marsh, Phoenix, AZ (US)

(73) Assignee: Marsh and Wang Medical Systems, LLC, Peoria, AZ (US)

(21) Appl. No.: 17/314,903

(22) Filed: May 7, 2021

Related U.S. Application Data
(63) Continuation-in-part of application No. 16/397,365, filed on Apr. 29, 2019.

(51) Int. Cl. A61K 31/5513; A61K 31/08; A61K 31/515; A61K 9/00

(52) U.S. Cl. CPC A61K 31/5513; A61K 9/0019; A61K 31/515; A61K 31/08

(57) ABSTRACT

A method for treating non-refractory status epilepticus includes administering together before onset of refractory status epilepticus, a therapeutically effective amount of a combination of halothane or a flurane, an anti-seizure benzodiazepine, and a barbiturate anti-convulsant. The flurane may selected from one or more of isoflurane, desflurane and sevoflurane. The anti-seizure benzodiazepine may be diazepam and the barbiturate anti-convulsant may be phenobarbital.

### CROSS-REFERENCE TO RELATED APPLICATIONS

This application is a continuation-in-part of U.S. patent application Ser. No. 16/397,365, filed Apr. 29, 2019, which is hereby incorporated by reference in its entirety.

### TECHNICAL FIELD

The present invention relates to seizure control compositions and methods; more particularly, to compositions and methods for the treatment of status epilepticus; and still more particularly, to compositions and methods for the treatment of status epilepticus comprising a three-drug combination.

### BACKGROUND OF THE INVENTION

Terrorism, and other international conflicts, makes the use of chemical warfare agents against civilians and/or military personnel a concern that requires adequate preparation. Nerve agents used in chemical warfare often lead to organophosphate poisoning. Exposure to nerve agents such as G-Series agents (i.e tabun (GA), sarin (GB), cyclosarin (GF), soman (GD)) and V-Series agents (i.e VX gas) results in a cholinergic crisis. Specifically, the inhibition of acetylcholinesterase (AChE), which is the primary metabolic enzyme of the cholinergic neurotransmitter acetylcholine. The resulting buildup of acetylcholine leads to an excessive release of the excitatory neurotransmitter glutamate from neuronal cells. This abundance of glutamate causes hyperexcitability in the brain. One of the consequences of this hyperexcitability can be status epilepticus (SE).

SE is a neurological emergency defined as either continuous seizure activity for greater than 30 minutes or recurrent seizure activity without a return to a baseline between events. SE is independently associated with high mortality and morbidity rates. SE needs to be treated immediately and effectively in order to prevent adverse outcomes including cognitive disorders, subsequent epilepsy and even death. The release of a chemical nerve agent in a civilian and/or military setting would result in nearby treatment facilities being overwhelmed with a significant number of victims presenting clinical signs of SE. While the above description is directed toward exposure to chemical agents during warfare, it should be noted that SE can occur without any known cause.

Exposure by any route is considered extremely neurotoxic. If an agent were inhaled, the estimated LCₜ50 ranges from 10 mg-min/m³ for VX to 40 mg-min/m³ for GA in any exposed population. If an agent were to come in direct contact with an individual's skin, one drop (40-50 μl) of VX can be fatal; while 1-10 mL of GA, GB, or GD can be fatal. The onset and severity of symptoms are dependent upon the concentration of the agent and route of exposure.

Current treatments in the field include military Mark|NAAK kits (Nerve Agent Antidote Kit) which contain autoinjectors with atropine and pralidoxime chloride (2-PAM). Atropine may treat seizures but only in a very narrow time window, i.e. within 5 minutes of exposure, which is mainly important for the prevention of systemic effects of the nerve agent (i.e. muscle contractions, excessive production of mucous, tears, saliva and sweat). 2-PAM, an oxime for disassociating the nerve agent from the cholinesterase molecule, does not have a theraputic effect on SE. Thus, the Mark|NAAK kits are ineffective in arresting and treating status epilepticus.

A separate autoinjector of the benzodiazepine, diazepam (DZP), is also available but studies of SE have shown that benzodiazepines alone will not be effective in up to 40% of cases. This 40% of cases are medically considered to be refractory status epilepticus (RSE).

RSE cases markedly complicate the logistics of acute treatment. Currently, there is no way to differentiate on presentation which cases will be responsive to benzodiazepine treatment versus those that will become refractory. Triaging could only be done after DZP is given and sufficient time is allowed to distinguish between the two populations. Current treatment protocols would then require placement of the RSE victims in barbiturate/anesthetic coma for hours/days to abate ictal activity. With mass nerve gas exposures, this would quickly saturate available intensive care unit resources to maintain such cases. Moreover, mortality, despite such RSE treatments, remains at 23%—although SE induced by nerve gas may result in higher rates. Additionally, survivors are more likely to have cognitive declines (85% of RSE versus 61% of responsive SE) and post-SE epilepsy (87.5% versus 22%).

Thus, what is needed is a more effective first-line SE treatment form that indiscriminately aborts responsive and refractory SE cases. This treatment would also have a favorable safety profile with minimal side effects, improve overall mortality rates, and minimize post-SE neurological deficits and de novo epilepsy cases.

### SUMMARY OF THE INVENTION

Briefly described, a method for treating non-refractory status epilepticus comprises administering together, before onset of refractory status epilepticus, a therapeutically effective amount of a combination of halothane or a flurane, an anti-seizure benzodiazepine, and a barbiturate anti-convulsant. The flurane may selected from one or more of isoflurane, desflurane and sevoflurane. The anti-seizure benzodiazepine may be diazepam and the barbiturate anti-convulsant may be phenobarbital. In accordance with an aspect of the present invention, the flurane may have a concentration between about 0.1% and about 5%, and may be administered via one or more of inhalation, subcutaneous injection, oral ingestion, intravenous injection, intramuscular injection, intraperitoneal injection and transdermal absorption.

### BRIEF DESCRIPTION OF THE DRAWINGS

The above-mentioned and other features are advantages of this invention, and the manner of attaining them, will become apparent and be better understood by reference to the following description of the invention in conjunction with the accompanying drawings, wherein:

FIG. 1 is a surgery diagram for placing EEG electrodes within a rat brain;

FIGS. 2A-2F show representative EEG traces from normal baseline through Stage 1, Stage 2, Stage 3, Stage 4 and Stage 5 of a seizure progression.

FIG. 3 shows EEG traces of two rats following induction of status epilepticus (SE);

FIG. 4 shows EEG traces for the same rats shown in FIG. 3, with rat 1 having been administered a three-component drug regimen in accordance with the present invention;

FIG. 5 shows EEG traces for the same rats shown in FIGS. 3 and 4, with both rats having been administered the three-component drug regimen;

FIG. 6 shows EEG traces for the same rats shown in FIGS. 3-5, two days after induction of SE and administration of the three-component drug regimen; and

FIG. 7 shows side-by-side EEG traces for two rats, with one rat receiving the standard two drug treatment and the other rat receiving an exemplary three-component drug regimen in accordance with the present invention.

### DETAILED DESCRIPTION

As described above, a typical treatment of SE may include administration of a combination of two standard antiepilepsy drugs (AEDs), diazepam (DZP) and phenobarbital (PB). Alternatively, such as in a clinical setting, DZP may be first administered to abort the ictal activity. If DZP alone is ineffective, PB may then be administered sequentially. In either event, these AEDs control the immediate onset of SE only about 60% of the time. The remaining roughly 40% of cases are referred to as refractory SE (RSE). In these instances, patients are at higher risk for lower cognitive function, recurrent unprovoked seizures (i.e. epilepsy), and death.

In accordance with an aspect of the present invention, a treatment for status epilepticus (SE) comprises administering a therapeutically effective combination of a flurane, an anti-seizure benzodiazepine, and a barbiturate anti-convulsant. The flurane may be selected from one or more of isoflurane, desflurane and sevoflurane, and in accordance with one aspect, is isoflurane. The flurane may be administered via any suitable delivery method, including but not limited to through inhalation, subcutaneous injection, oral ingestion, intravenous injection, intramuscular injection, intraperitoneal injection and transdermal absorption. The concentration of the flurane may range from about 0.1% to about 5%, more particularly about 1.5% to about 3.5%, and still more particularly about 2% to about 3%. When inhaled, the remainder of the inhalation gas is typically oxygen. The anti-seizure benzodiazepine may be selected from one or more of chlorazepate, clobazam, clonazepam, diazepam, levetiracetam, lorazepam, midazolam or nitrazepam, and in one aspect is diazepam (DZP). The barbiturate anti-convulsant may be selected from one or more of phenobarbital, mephobarbital or primidone, and in one aspect is phenobarbital (PB).

As set forth in the below example, a treatment of status epilepticus comprises administration of a therapeutically effective amount of the three-drug combination. By way of example and without limitation thereto, one example of an effective three-drug combination includes DZP: PB: Isoflurane.

### EXPERIMENTAL

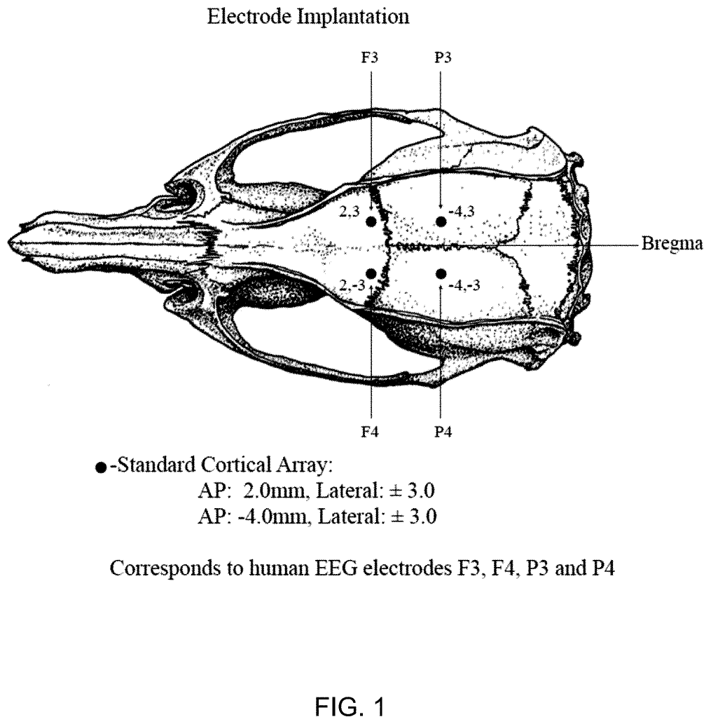

Adult male Sprague Dawley rats (approximately 200-250 grams) underwent surgery to have epidural screw electrodes implanted in the skull for EEG recording to detect electrographic changes induced by seizures. See locations F3, F4, P3, P4 in FIG. 1. The rats were housed singly after surgery, with food and water available ad libitum, with a 24 hour diurnal light cycle maintained, with lights on from 0700 to 1900 each day. All animal procedures were conducted in accordance with National Institute of Health's (NIH) Guide for the Care and Use of Laboratory Animals: Eighth Edition (2011), The Association for Assessment and Accreditation of Laboratory Animal Care Guidelines and the Institutional Animal Care and Use Committee.

Treatment response and concentrations were based on a single animal model for SE. Rate and intensity of the development of chronic epilepsy was determined following the induction and treatment of SE. The rats were fed a mush containing rat biscuits and water and were continuously monitored with EEG recording for 12 weeks to detect the development of chronic epilepsy. The number, frequency, and duration of seizures was recorded for each rat every day. This time duration was chosen to detect progressive long term changes as the result of the treatments.

A lithium-pilocarpine protocol was used to generate Generalized Convulsive Status Epilepticus (GCSE) in the rats. Tylenol (1-2 mg per ml) was added to drinking water the day before surgery and for three days post-operatively. One week after electrode implantation surgery, a baseline EEG was recorded for 15 minutes. Status epilepticus was then induced by an intraperitoneal (IP) injection of lithium chloride (3 mmol/kg) followed by subcutaneous (SC) injection of pilocarpine (30 mg/kg) 20-24 hours later. Following injection of pilocarpine, the EEG of each rat was monitored continuously by being placed in a recording cage and connected to a clinical EEG machine by a flexible cable suspended from the top of the cage. The cage was equipped with an interposed commutator system to allow the rats to turn freely without twisting the cable.

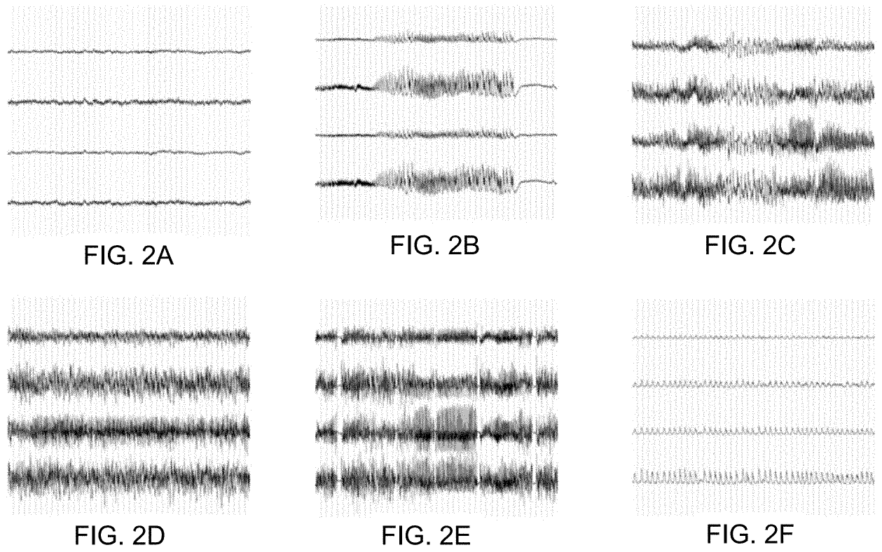

Turning now to FIG. 2, a progression of lithium-pilocarpine induced SE is shown. FIG. 2A shows baseline EEG traces from electrodes F4, P4, F3, P3 shown arranged from top to bottom prior to injection with pilocarpine. FIG. 2B shows EEG traces from the same electrodes 23 minutes after pilocarpine injection. As can be seen, the rat is in the initial stages of a seizure episode (designated Stage 1) which is generally indicated physically through tail twitches and head bobbing. FIG. 2C shows EEG traces 29 minutes post pilocarpine injection as the seizure progresses to Stage 2 and the rat begins to exhibit more severe physical manifestations including tremors and shaking. FIG. 2D shows EEG traces 37 minutes post pilocarpine injection with the rat in Stage 3, or fully ictal. Stage 3 is the most violent stage of the seizure and includes physical signs such as violent flopping of the rat and foaming at the mouth. FIG. 2E shows EEG traces taken 58 minutes post pilocarpine injection. As can be seen in the traces, breaks within the ictal seizure indicate that the rat has entered Stage 4 wherein neurochemicals in the brain are becoming exhausted and neurological damage is occurring. Finally, FIG. 2F shows EEG traces more than 2 hours post pilocarpine injection. The rat has entered Stage 5 wherein death is imminent without immediate medical attention. And, even if medical attention were successfully administered, the rat will have severe cognitive and memory impairments.

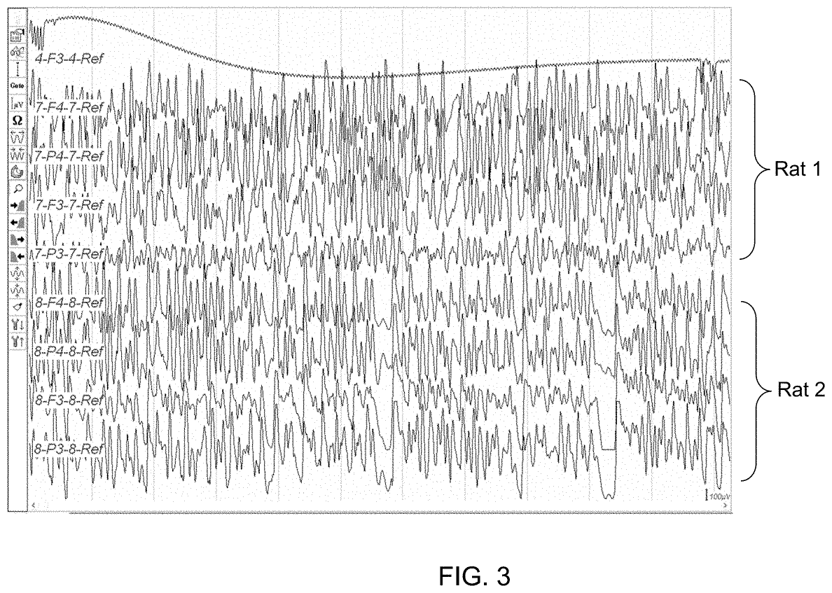

With reference to FIGS. 3-6, an episode of SE was chemically induced and the rats were then treated with a systemic injection of the standard antiepileptic drugs DZP and PB in combination with inhaled isoflurane/oxygen in accordance with the present invention to abort later stages of RSE, as measured by time and EEG characteristics. For example, and without limitation thereto, each rat was administered 10 mg/kg DZP, 25 mg/kg PB and 2-3% isoflurane concentration after each animal progressed to complete ictal pattern of SE on the EEG (Stage 3 as defined above with regard to FIG. 2D). Each animal was continuously monitored for the next 4 to 6 hours. Following the acute experiment, each rat was chronically monitored for over 3 months.

With specific reference to FIG. 3, EEG traces of two rats (rat 1 and rat 2) following lithium-pilocarpine induction of status epilepticus (SE) are shown. Traces 1-4 are for rat 1, while traces 5-8 are for rat 2. As can be seen, each rat is in a completely ictal state of SE (Stage 3). It should be noted that Stage 3 was selected as the stage for medical intervention as Stage 3 is the most violent portion of the seizure. Moreover, most patients received within a hospital emergency department for seizure will likely have progressed to at least stage 3 before arrival.

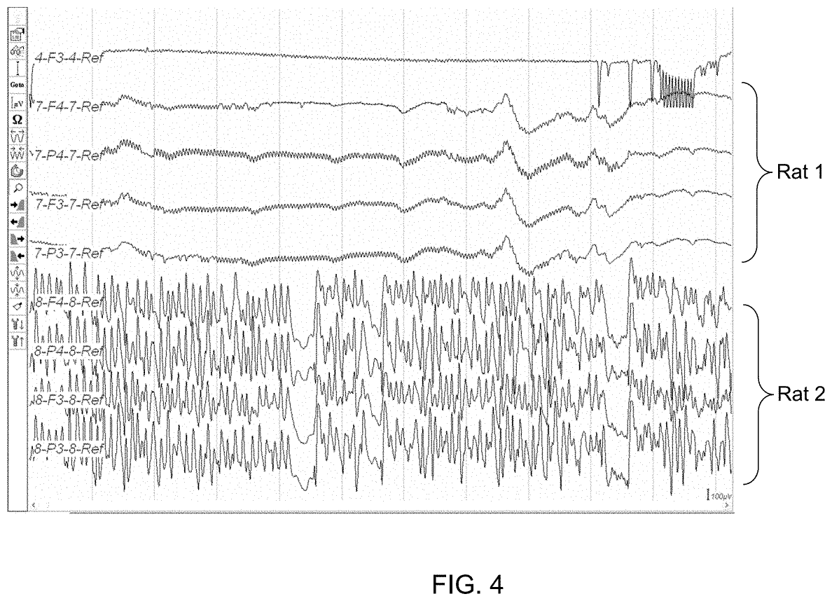

FIG. 4 shows traces for the same rat 1 and rat 2, with rat 1 having been administered the three-component drug regimen in accordance with the present invention, consisting of 10 mg/kg DZP, 25 mg/kg PB and 2-3% isoflurane in oxygen. It should be understood by those skilled in the art that the above composition is exemplary and is not to be seen as limiting in any way. It should be further noted that alternative concentrations or ranges of concentrations may be utilized provided such compositions are therapeutically effective in accordance with the present invention.

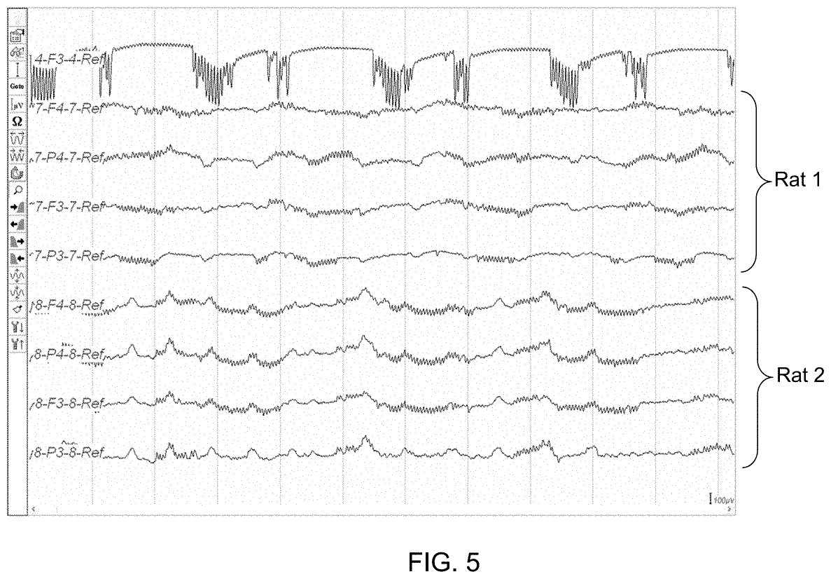

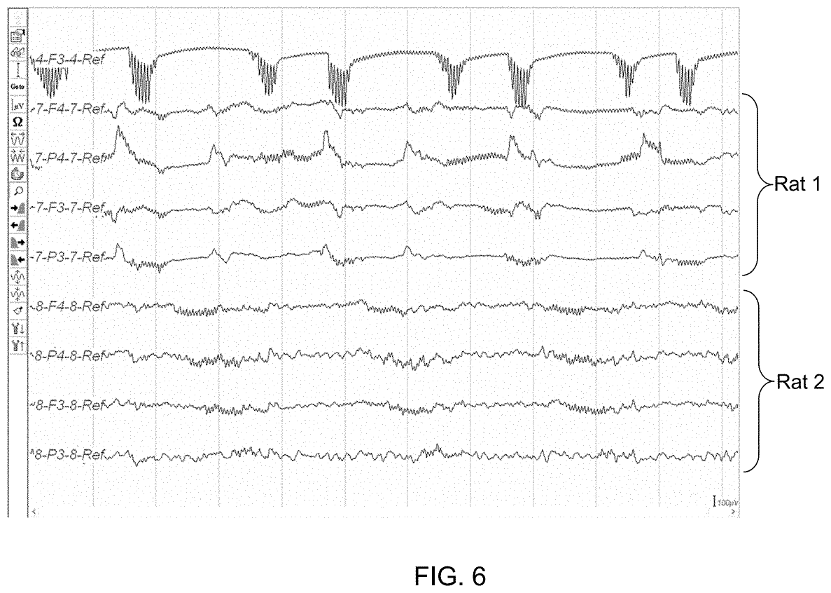

As can be seen in FIG. 4, the EEG traces from rat 1 show a return to normal EEG patterns (baseline) while rat 2 continues in the completely ictal state (Stage 3). However, as shown in FIG. 5, EEG traces for the same rats 1 and 2 show each rat returning to baseline following administration of the three-component drug regimen in accordance with the present invention. Finally, FIG. 6 shows EEG traces two days after induction of SE and administration of the three-component drug regimen for rats 1 and 2. Each rat continues to exhibit normal EEG (baseline) activity with no evidence of seizures over the course of approximately 48 hours. Rats 1 and 2 were then followed for a further 17 weeks with no recurrence of spontaneous seizures or other indications of epilepsy.

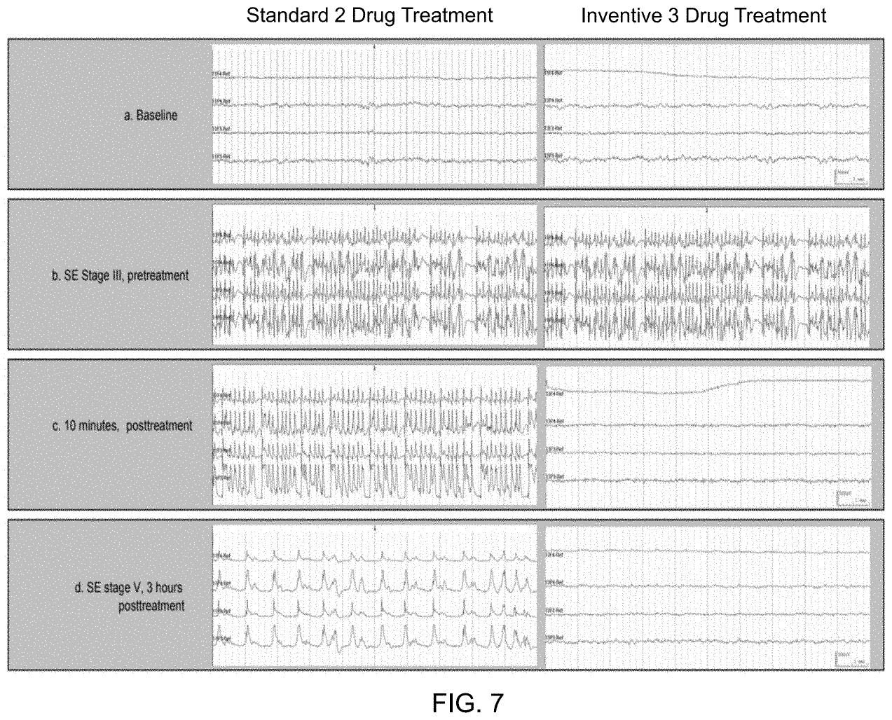

With reference to FIG. 7, a side-by-side comparison of Stage 3 rats given the standard two drug AED treatment (left-hand side) and the inventive three drug treatment (right-hand side) is shown. As can be seen, the standard two drug treatment does not affect the observed ictal EEG morphology as shown in FIGS. 2D-2F. Rather, the rats continue uninterrupted in the ictal pattern (Stage 3, top left) until a point when the brain resources begin to become exhausted (Stage 4, middle left). It is then that flatline “breaks” in between the ictal pattern become wider and more frequent over the next few hours. The flatline breaks then become continuous, marked with an occasional sharp spike (Stage 5, bottom left). These spikes are known clinically as PED's or periodic epileptiform discharges. The presence of PED's indicates that death is imminent and the rodent will be deceased a total of 4.5-5 hours following the pilocarpine injection, despite receiving the standard two drug AED treatment. The rare rat (less than 10%) that did survive (only after excessive medical intervention) would develop epilepsy about 2 weeks following the acute experiment and suffer anywhere between 30-80 seizures a week. These rats also exhibited significant cognitive impairments. In contrast, as shown on the right-hand side of FIG. 7, rats that were administered the three drug treatment in accordance with the present invention aborted the SE (in less than two minutes) and returned to normal baseline EEG traces. These rats also do not develop epilepsy or suffer from future seizure events.

While the above was described with reference to a lithium-pilocarpine induction model, it should be noted that similar ictal patterns may be caused by other models/chemical agents, with complete ictal activity progressing at different rates/times.

While the invention has been described by reference to various specific embodiments, it should be understood that numerous changes may be made within the spirit and scope of the inventive concepts described. Accordingly, it is intended that the invention not be limited to the described embodiments, but will have full scope defined by the language of the following claims.

What is claimed is:

1. A method for treating non-refractory status epilepticus comprising, administering together before onset of refractory status epilepticus a therapeutically effective amount of a combination of halothane or a flurane, an anti-seizure benzodiazepine, and a barbiturate anti-convulsant.

2. The method in accordance with claim 1 wherein the flurane is selected from one or more of isoflurane, desflurane and sevoflurane.

3. The method cage in accordance with claim 2 wherein the flurane is isoflurane.

4. The method in accordance with claim 1 wherein the anti-seizure benzodiazepine is selected from one or more of chlorazepate, clobazam, clonazepam, diazepam, levetiracetam, lorazepam, midazolam or nitrazepam.

5. The method in accordance with claim 4 wherein the anti-seizure benzodiazepine is diazepam.

6. The method in accordance with claim 1 wherein the barbiturate anti-convulsant is selected from one or more of phenobarbital, mephobarbital or primidone.

7. The method of claim 6 wherein the barbiturate anti-convulsant is phenobarbital.

8. The method in accordance with claim 1 wherein the flurane has a concentration between about 1% and about 5%.

9. The method in accordance with claim 1 wherein the flurane is administered via one or more of inhalation, subcutaneous injection, oral ingestion, intravenous injection, intramuscular injection, intraperitoneal injection and transdermal absorption.

10. A method for treating a patient exposed to a chemical warfare agent, the method comprising:
a) providing a first syringe preloaded with an anti-seizure benzodiazepine and a barbiturate anti-convulsant;
b) providing a container preloaded with halothane or a flurane;
c) injecting the patient with the first syringe; and
d) administering the halothane or flurane to the patient immediately after step c).

11. The method in accordance with claim 10 wherein the patient performs steps c) and d).

12. The method in accordance with claim 11 wherein the patient is a military personnel and wherein the halothane or flurane is administered via inhalation using a military issue gas mask.
